(19) United States
(12) Patent Application Publication
(10) Pub. No.: US 2014/0261424 A1
(43) Pub. Date: Sep. 18, 2014

(54) METHODS AND SYSTEMS FOR PHASE SHIFTED PRESSURE VENTILATION

(71) Applicant: COVIDIEN LP, Boulder, CO (US)

(72) Inventors: Peter Doyle, Vista, CA (US); Gardner Kimm, Carlsbad, CA (US)

(73) Assignee: Covidien LP, Boulder, CO (US)

(21) Appl. No.: 13/799,987

(22) Filed: Mar. 13, 2013

(51) Int. Cl. A61M 16/00; A61M 16/06; A61M 16/08; A61M 16/04

(52) U.S. Cl. CPC A61M 16/0051; A61M 16/0057; A61M 16/04; A61M 16/06; A61M 16/0875
USPC 128/204.23

(57) ABSTRACT

This disclosure describes systems and methods for providing pressure ventilation with a shifted pressure profile during ventilation of a patient. The disclosure describes a novel breath type that phase shifts the rise time of the delivered pressure profile to improve patient comfort and patient-ventilator synchrony.

### INTRODUCTION

Medical ventilator systems have long been used to provide ventilatory and supplemental oxygen support to patients. These ventilators typically comprise a source of pressurized oxygen which is fluidly connected to the patient through a conduit or tubing. As each patient may require a different ventilation strategy, modern ventilators can be customized for the particular needs of an individual patient. For example, several different ventilator modes or settings have been created to provide better ventilation for patients in various different scenarios.

### Phase Shifted Pressure Ventilation

This disclosure describes systems and methods for providing pressure ventilation with a shifted pressure profile during ventilation of a patient. The disclosure describes a novel breath type that phase shifts the rise time of the delivered pressure profile to improve patient comfort and patient-ventilator synchrony.

In part, this disclosure describes a method for ventilating a patient with a ventilator. The method includes:

receiving a set pressure;

determining a pressure profile based at least on the received set pressure;

phase shifting the pressure profile to form a shifted pressure profile; and

delivering the shifted pressure profile to a patient.

Yet another aspect of this disclosure describes a ventilator system that includes a pressure generating system, a ventilation tubing system, a support module, and a phase shift module. The pressure generating system is adapted to generate a flow of breathing gas. The ventilation tubing system includes a patient interface for connecting the pressure generating system to a patient. The support module determines a pressure profile based at least on the received set pressure. The phase shift module phase shifts the pressure profile determined by the support module to form a shifted pressure profile. The pressure generating system delivers the shifted pressure profile to the patient.

The disclosure further describes a computer-readable medium having computer-executable instructions for performing a method for ventilating a patient with a ventilator. The method includes:

receiving a set pressure;

repeatedly determining a pressure profile based at least on the received set pressure;

repeatedly phase shifting the pressure profile to form a shifted pressure profile; and

repeatedly delivering the shifted pressure profile to a patient.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the drawings.

It is to be understood that both the foregoing general description and the following detailed description are exemplary and explanatory and are intended to provide further explanation of the claims.

### BRIEF DESCRIPTION OF THE DRAWINGS

The following drawing figures, which form a part of this application, are illustrative of embodiments of systems and methods described below and are not meant to limit the scope of the disclosure in any manner, which scope shall be based on the claims.

FIG. 1 illustrates an embodiment of a schematic diagram of a ventilator.

FIG. 2 illustrates an embodiment of a communication schematic diagram between ventilator components.

FIG. 3 illustrates an embodiment of a method for ventilating a patient on a ventilator during a first breath in a pressure targeted breath type.

FIG. 4 illustrates an embodiment of a graph of inspiratory pressure profiles delivered to a patient being ventilated with a medical ventilator.

FIG. 5 illustrates an embodiment of a graph of adjusted inspiratory pressure profiles delivered to a patient being ventilated with a medical ventilator.

FIG. 6 illustrates an embodiment of a graph of adjusted inspiratory pressure profiles delivered to a patient being ventilated with a medical ventilator.

FIG. 7 illustrates an embodiment of a graph of adjusted inspiratory pressure profiles delivered to a patient being ventilated with a medical ventilator.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. A person of skill in the art will understand that the technology described in the context of a medical ventilator for human patients could be adapted for use with other systems such as ventilators for non-human patients and general gas transport systems.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen is supplied to the patient at desired pressures and rates. Ventilators capable of operating independently of external sources of pressurized air are also available.

While operating a ventilator, it is desirable to control the percentage of oxygen in the gas supplied by the ventilator to the patient. Further, as each patient may require a different ventilation strategy, modern ventilators can be customized for the particular needs of an individual patient. For example, several different ventilator breath types have been created to provide better ventilation for patients in various different scenarios.

Pressure breath types, such as pressure support (PS) ventilation or pressure control (PC), delivered to actively breathing patients may have an adjustable rise time to modify the rate which pressure increases in an effort to match ventilator delivery with patient inspiratory demand. However, despite the ability to adjust the slope of the pressure rise, the profile of the patient's inspiratory effort is often not well matched to that of the profile delivered by the ventilator.

For example, studies have shown that patients with specific disease states, such as chronic obstructive pulmonary disease (COPD), treated with noninvasive ventilation were able to unload the respiratory muscles better when the rise time was set to increase pressure at a faster rate, but this result was also associated with higher leakage and greater patient discomfort. One possible reason for the high leakage and greater patient discomfort is that when the rise time is set to a steeper slope, the higher pressure and the flows that occur early in the inspiratory phase result in a reflexive glottal restriction or gagging reflex. The block of flow into the lungs results in increased leak past the patient interface (e.g., mask) and patient discomfort. Conversely, patients that better tolerate a slower rise time setting may find that there is not sufficient time to reach the set pressure level, resulting in less than adequate volume delivery.

Accordingly, the current disclosure describes systems and methods for phase shifting the pressure profile of the pressure based ventilation. The adjustable phase shift delays delivering the determined pressure profile for a predetermined amount of time. During the phase shift the ventilator delays the delivery of the determined pressure profile by not increasing the pressure or by utilizing a diminished rise time for the pressure delivery. The phase shift allows the lungs to partially inflate. Once the lungs are partially inflated, the ventilator changes to the faster rise time of the determined pressure profile without the associated problems of increased leak and patient discomfort as described above.

FIG. 1 is a diagram illustrating an embodiment of an exemplary ventilator 100 connected to a human patient 150. FIG. 2 illustrates an embodiment of a schematic communication diagram between different ventilator components. Ventilator 100 includes a pneumatic system 102 (also referred to as a pressure generating system 102) for circulating breathing gases to and from patient 150 via the ventilation tubing system 130, which couples the patient 150 to the pneumatic system 102 via an invasive (e.g., endotracheal tube, as shown) or a non-invasive (e.g., nasal mask) patient interface 180.

Ventilation tubing system 130 (or patient circuit 130) may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, pneumatic system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 and the expiratory module 108 to provide a gas source for ventilatory support via inspiratory limb 132.

The inspiratory module 104 is configured to deliver gases to the patient 150 according to prescribed ventilatory settings. In some embodiments, inspiratory module 104 is configured to provide ventilation according to various breath types, e.g., via pressure-control, pressure-control, pressure assist, or via any other suitable breath types.

The expiratory module 108 is configured to release gases from the patient's lungs according to prescribed ventilatory settings. Specifically, expiratory module 108 is associated with and/or controls an expiratory valve for releasing gases from the patient 150.

The ventilator 100 may also include one or more sensors 107 communicatively coupled to ventilator 100. The sensors 107 may be located in the pneumatic system 102, ventilation tubing system 130, and/or on the patient 150. The embodiment of FIG. 1 illustrates a sensor 107 in pneumatic system 102.

Sensors 107 may communicate with various components of ventilator 100, e.g., pneumatic system 102, other sensors 107, processor 116, support module 117, phase shift module 118, and/or any other suitable components and/or modules. In one embodiment, sensors 107 generate output and send this output to pneumatic system 102, other sensors 107, processor 116, support module 117, phase shift module 118, and/or any other suitable components and/or modules. Sensors 107 may employ any suitable sensory or derivative technique for monitoring one or more patient parameters or ventilator parameters associated with the ventilation of a patient 150. Sensors 107 may detect changes in patient parameters indicative of patient triggering, for example. Sensors 107 may be placed in any suitable location, e.g., within the ventilatory circuitry or other devices communicatively coupled to the ventilator 100. Further, sensors 107 may be placed in any suitable internal location, such as, within the ventilatory circuitry or within components or modules of ventilator 100. For example, sensors 107 may be coupled to the inspiratory and/or expiratory modules for detecting changes in, for example, circuit pressure and/or flow. In other examples, sensors 107 may be affixed to the ventilatory tubing or may be embedded in the tubing itself. According to some embodiments, sensors 107 may be provided at or near the lungs (or diaphragm) for detecting a pressure in the lungs. Additionally or alternatively, sensors 107 may be affixed or embedded in or near wye-fitting 170 and/or patient interface 180. For example, one or more sensors 107 may generate output regarding inspiration flow and/or inspiration pressure. Additionally, in some embodiments, the ventilator 100 may derive inspiratory demand of the patient 150 based on inspiratory flow and/or inspiratory pressure generated sensor output. In some embodiments, the ventilator 100 monitors resistance and/or compliance based on sensor output or derived sensor output. Indeed, any sensory device useful for monitoring changes in measurable parameters during ventilatory treatment may be employed in accordance with embodiments described herein.

As should be appreciated, with reference to the Equation of Motion, ventilatory parameters are highly interrelated and, according to embodiments, may be either directly or indirectly monitored. That is, parameters may be directly monitored by one or more sensors 107, as described above, or may be indirectly monitored or estimated/calculated using a model, such as a model derived from the Equation of Motion (e.g., Target Airway Pressure(t)=Eₚ∫Qₚdt+QₚRₚ−Patient Effort(t)).

The pneumatic system 102 may include a variety of other components, including mixing modules, valves, tubing, accumulators, filters, etc. Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.).

In one embodiment, the operator interface 120 of the ventilator 100 includes a display 122 communicatively coupled to ventilator 100. Display 122 provides various input screens, for receiving clinician input, and various display screens, for presenting useful information to the clinician. In one embodiment, the display 122 is configured to include a graphical user interface (GUI). The GUI may be an interactive display, e.g., a touch-sensitive screen or otherwise, and may provide various windows and elements for receiving input and interface command operations. Alternatively, other suitable means of communication with the ventilator 100 may be provided, for instance by a wheel, keyboard, mouse, or other suitable interactive device. Thus, operator interface 120 may accept commands and input through display 122. Display 122 may also provide useful information in the form of various ventilatory data or regarding the physical condition of a patient 150. The useful information may be derived by the ventilator 100, based on data collected by a processor 116, and the useful information may be displayed to the clinician in the form of graphs, wave representations, pie graphs, text, or other suitable forms of graphic display. For example, patient data may be displayed on the GUI and/or display 122. Additionally or alternatively, patient data may be communicated to a remote monitoring system coupled via any suitable means to the ventilator 100. In one embodiment, the display 122 may display one or more of a phase shift, a pressure profile, a predetermined amount of time, a diminished rise time, an inspiratory demand, a set pressure, an adjusted rise time, a rise time, a shifted pressure profile, an activation of phase shifting, a patient disease state, a patient drive during P₁₀₀, compliance, resistance, and/or a flow waveform of a non-pressure supported breath.

Controller 110 may include memory 112, one or more processors 116, storage 114, and/or other components of the type commonly found in command and control computing devices. Controller 110 may further include a support module 117 and/or a phase shift module 118 as illustrated in FIG. 1. In alternative embodiments, the support module 117 and/or the phase shift module 118 are located in other components of the ventilator 100, such as the pressure generating system 102 (also known as the pneumatic system 102).

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

The pneumatic system 102 provides ventilation via a form of pressure ventilation. Pressure-targeted breath types are provided by regulating the pressure delivered to the patient 150 in various ways. Pressure ventilation may include a pressure-support (PS), a proportional assist (PA), tube compensation (TC), or a pressure-control (PC) breath type, for example. The proportional assist (PA) breath type provides pressure in proportion to the instantaneous patient effort during spontaneous ventilation and is based on the equation of motion. Pressure ventilation is accomplished by setting a target or prescribed pressure for delivery to the patient 150. During pressure ventilation, inspiration time may be determined based on normal respiratory and compliance values and on the patient's ideal body weight. According to some embodiments, an exhalation time may be determined based on normal respiratory and compliance values and based on the patient's ideal body weight. A respiratory rate (RR) setting may also be determined and configured. For a non-triggering patient, the set RR controls the timing for each inspiration. For a triggering patient, the RR setting applies if the patient 150 stops triggering for some reason and/or patient triggering drops below a threshold RR level.

According to embodiments, during pressure ventilation, the ventilator 100 may maintain the same pressure waveform at the mouth, P_{awo}, regardless of variations in lung or airway characteristics, e.g., respiratory compliance and/or respiratory resistance. However, the volume and flow waveforms may fluctuate based on lung and airway characteristics. As noted above, pressure delivered to the upper airway creates a pressure gradient that enables gases to flow into a patient's lungs. The pressure from which a ventilator 100 initiates inspiration is termed the end-expiratory pressure (EEP) or “baseline” pressure. This pressure may be atmospheric pressure (about 0 cm H₂O), also referred to as zero end-expiratory pressure (ZEEP). However, commonly, the baseline pressure may be positive, termed positive end-expiratory pressure (PEEP). Among other things, PEEP may promote higher oxygenation saturation and/or may prevent alveolar collapse during exhalation. Under pressure-cycled ventilation, upon delivering the prescribed pressure the ventilator 100 may initiate exhalation.

In some embodiments, the pneumatic system 102 deliver gases to the patient 150 according to the pressure control (PC) breath type. PC allows a clinician to select a pressure to be administered to a patient 150 during a mandatory breath. When using the PC breath type, a clinician sets a desired pressure, inspiratory time, and respiratory rate for a patient 150. These variables determine the pressure of the gas delivered to the patient 150 during each mandatory breath inspiration. The mandatory breaths are administered according to the set respiratory rate.

For the PC breath type, when the inspiratory time is equal to the prescribed inspiratory time, the ventilator 100 may initiate exhalation. Exhalation lasts from the end of inspiration until the next inspiration. Upon the end of exhalation, another PC mandatory breath is given to the patient 150.

During PC breaths, the ventilator 100 may maintain the same pressure waveform at the mouth, regardless of variations in lung or airway characteristics, e.g., respiratory compliance and/or respiratory resistance. However, the volume and flow waveforms may fluctuate based on lung and airway characteristics.

In some embodiments, PC may also be delivered for triggering patients. When PC is delivered with triggering, the breath period (i.e. time between breaths) is a function of the respiratory rate of the patient 150. The ventilator 100 will trigger the inhalation based upon the respiratory rate setting or the patient trigger effort, but cycling to exhalation will be based upon elapsed inspiratory time. The inspiratory time is set by the clinician. The inspiratory flow is delivered based upon the pressure setting and patient physiology. Should the patient 150 create an expiratory effort in the middle of the mandatory inspiratory phase, the ventilator 100 will respond by reducing flow. If no patient effort is detected, the ventilator 100 will deliver another mandatory breath at the predetermined respiratory rate.

In some embodiments, the pneumatic system 102 of the ventilator 100 delivers gases to the patient 150 according to pressure-support (PS) ventilation. PS ventilation is a form of assisted ventilation and may be utilized during either a mandatory breath or a spontaneous breath. When the ventilator 100 senses patient inspiratory effort, the ventilator 100 provides a constant pressure during inspiration. The pressure may be set and adjusted by the clinician. The patient 150 controls the rate, inspiratory flow, and inspiratory time. The ventilator 100 then adjusts the volume over several breaths to achieve the set pressure. When the machine senses a decrease in flow, or determines that inspiratory time has reached a predetermined limit, the ventilator 100 determines that inspiration is ending. When delivered as a spontaneous breath, expiration in PS lasts from a determination that inspiration is ending until the ventilator 100 senses a patient effort to breath.

The support module 117 is configured to determine a pressure profile based at least on the received set pressure. The pressure profile is the slope or the rise time of the pressure provided to the patient 150 over time during inspiration. In other words, the pressure profile is the inspiratory pressure waveform delivered to the patient 150. For example, FIG. 5 illustrates an embodiment of a graph showing three different rise times delivered to patient 150 over inspiration time during a pressure based ventilation. The received set pressure is input or selected by the operator into the ventilator 100. The support module 117 determines a pressure profile that is configured to deliver the set pressure to patient 150 during inspiration. Inspiratory time for the pressure profile, as discussed above may be determined based on monitored resistance and compliance values and on the patient's ideal body weight or based on a set respiration rate. Accordingly, in some embodiments, the support module 117 determines the pressure profile based on a received respiration rate and/or ideal body weight of the patient 150. The received respiration rate and/or the received ideal body weight is input or selected by the operator into the ventilator 100. The support module 117 sends the determined pressure profile to the other ventilator components, such as pressure generating system 102, inspiratory module 104, the processor 116, the phase shift module 118, and/or any other suitable ventilator component for delivering the determined pressure profile.

In an effort to improve patient comfort and leak, previously utilized ventilators have adjusted rise time in attempt to mitigate these problems. In some embodiments, the support module 117 of the ventilator 100 determines an adjusted rise time based on generated sensor output. In some embodiments, the support module 117 of the ventilator 100 determines an adjusted rise time based on patient demand. The patient demand may be derived from generated sensor output, such as patient inspiratory flow and/or patient inspiratory pressure. In other embodiments, the ventilator 100 determines an adjusted rise time based on resistance or compliance. The resistance and/or compliance may be received and/or derived from generated sensor output. In these embodiments, the support module 117 determines the pressure profile based on the received set pressure and the determined adjusted rise time.

In an embodiment, where the rise time is adjusted based on patient drive an indication of a patient's respiratory drive is made by measuring the pressure generated in the first 100 milliseconds (ms) of inspiration (P₁₀₀). This calculation is derived from generated sensor output. The higher the P₁₀₀, the greater the respiratory drive. Typically, a high respiratory drive (e.g., P₁₀₀>6 cmH₂O) implies a higher inspiratory flow at an earlier portion of the inspiratory phase. Therefore, in this embodiment, patients exhibiting a high P₁₀₀ are treated with a faster rise time profile, such as a Rise Time A as illustrated in FIGS. 4-7. In this embodiment, patients with a lower P₁₀₀ (e.g., 2.0 cmH₂O or less) are treated with a slower rise time, such as Rise Time C as illustrated in FIGS. 4-7.

In patients with a low compliance and/or a high resistance, too fast of a rise time can cause the inspiratory phase to end early. This is the result of a pressure overshoot that causes the ventilator 100 to prematurely detect the end of inspiration. Accordingly, in embodiments, where the rise time is adjusted based on compliance or resistance, patient 150 with low compliance and/or high resistance (e.g., acute respiratory distress syndrome (ARDS)) are treated with a slower rise time, such as Rise Time C as illustrated in FIGS. 4-7.

However, adjusting the rise time does not remedy the problems discussed above of patient discomfort and/or leakage. For example, if a high rise time is utilized, as illustrated in with Rise Time A in FIG. 5, the patient 150 receives too much air to quickly often resulting in the reflexive glottal restriction or gagging reflex discussed above. If a low rise time is utilized, as shown in Rise Time C in FIG. 5, the patient 150 may not receive enough flow to meet their inspiratory demand and/or enough volume of gas leading to under-ventilation. A moderate rise time, as illustrated by Rise Time B in FIG. 5, may be comfortable at the early phase of inspiration but may also be uncomfortable for the patient at the latter stages of inspiration 150 causing the patient 150 to fight the delivered pressure ventilation.

As discussed above, the ventilator 100 also includes a phase shift module 118. The phase shift module 118 phase shifts the determined pressure profile from the support module 117 to form a shifted pressure profile. In some embodiments, the phase shift module 118 automatically phase shifts the determined pressure profile. In other embodiment, the phase shift module 118 phase shifts the determined pressure profile based on operator selection or input. For example, the operator may select and apply a phase shift button. In other embodiments, the operator may input the disease state, such as COPD, asthmatic, or congestive heart failure, of the patient 150 and the ventilator 100 may automatically activate the phase shift module 118 based on the entered disease state.

The phase shift is an adjustable delay of the delivery of the pressure profile determined by the support module 117 for a predetermined amount of time. In some embodiments, phase shift module 118 adjusts the phase shift applied to the pressure profile between breaths or after a predetermined number of breaths based on operator input and/or generated sensor output. In other embodiments, the phase shift module 118 adjust the phase shift applied to the pressure profile while being delivered to patient 150 or during the inspiratory phase breaths based on operator input and/or generated sensor output. For example, in some embodiments, the phase shift module 118 adjust the delay or the phase every computation cycle, such as every 5 ms based on operator input and/or generated sensor output.

While the phase shift discussed herein and as referenced in the FIGS. start just after the detection of an inspiration, the phase shift module 118 may implement a phase shift at any suitable point during an inspiration. For example, the phase shift module 118 may implement a phase shift at the end of an inspiration in order to increase patient comfort. In another example, the phase shift may be at the beginning of one breath and in the middle of the following breath.

In some embodiments, the phase shift module 118 determines the desired phase shift based on a function of delivered volume, inspiration time, a percentage of inspiration time, a percentage of delivered volume, and/or pressure reached.

In some embodiments, the phase shift includes not increasing the pressure delivered to the patient 150 for the entire predetermined amount of time as illustrated in FIG. 5. Accordingly, the ventilator 100 may deliver the set PEEP or no pressure to the patient 150 for the entire predetermined amount of time. In other embodiments, the phase shift includes increasing the pressure delivered to the patient 150 at a diminished rise time for the entire predetermined amount of time as illustrated in FIG. 6. In further embodiments, the phase shift includes during a first portion of the predetermined amount of time not increasing the pressure delivered to the patient 150 and in a consecutive second portion of the predetermined amount of time increasing the pressure delivered to the patient 150 at a diminished rise time for the as illustrated in FIG. 7.

As discussed above the inspiratory demand of patients may differ from ventilator delivery time as well as the delivered pressure slope. Providing a phase shift or a delay allows the lungs to partially inflate before the delivery of the pressure profile. Further, the phase shift delays the occurrence of peak flow rate until after the phase shift. Accordingly, once the lungs are partially inflated, the ventilator 100 switches to the faster rise time of the pressure profile without the associated problems of leak and gagging as discussed above. As discussed above, the phase shifting of the pressure profile forms the shifted pressure profile.

The predetermined amount of time is any suitable amount of time for delaying the delivery of the pressure profile. In some embodiments, the predetermined amount of time is any suitable amount of time for allowing the lungs to partially inflate enough to allow for the delivery of the pressure profile rise time without resulting in a gagging reflex. In some embodiments, the predetermined amount of time ranges from about 30 ms to 400 ms. In other embodiments, the predetermined amount of time ranges from about 100 ms to 300 ms. In further embodiments, the predetermined amount of time ranges from about 150 ms to 250 ms. In some embodiments, the predetermined amount of time is about 50 ms, 75 ms, 100 ms, 125 ms, 150 ms, 175 ms, 200 ms, 225 ms, 250 ms, 275 ms, 300 ms, 325 ms, 350 ms, 375 ms, or 400 ms.

In some embodiments, the predetermined amount of time of the phase shift is a set time applied to each breath. In other embodiments, the predetermined amount of time may vary or be dynamic from breath to breath. For example, the phase shift module 118 may determine a dynamic predetermined amount of time for each breath based on a function of delivered volume, inspiration time, a percentage of inspiration time, a percentage of delivered volume, and/or pressure reached.

The diminished rise time is a rise time that is less than the rise time for the pressure profile determined by the support module 117. In some embodiments, the diminished rise time is from about 20% to 95% less than the rise time for the pressure profile determined by the support module 117. In other embodiments, the diminished rise time is from about 30% to 80% less than the rise time for the pressure profile determined by the support module 117. In further embodiments, the diminished rise time is from about 50% to 80% less than the rise time for the pressure profile determined by the support module 117. In some embodiments, the diminished rise time is about 20%, 30%, 40%, 50%, 60%, 80% or 90% less than the rise time for the pressure profile determined by the support module 117.

The phase shift module 118 applies an adjustable phase shift based on operator input and/or generated sensor output. In some embodiments, the phase shift module 118 applies a phase shift based on operator input or selection. For example, the operator may select between a set of preprogrammed phase shifts or may enter a desired phase shift. In another example, the operator may input ventilator settings such as inspiratory time and/or patient parameters such as age, weight, sex, and/or disease state and the ventilator 100 may determine the proper phase shift based upon the operator entered information. In some embodiments, the operator may input an adjustment of the phase shift based on patient reaction. For example, the operator may input or select a phase shift and then watch to see if the patient reacts favorably to the phase shift. A positive patient reaction may be observed by a normal chest rise and fall (instead of a sudden chest jerk), by displaying a positive facial reaction, or by displaying a thumbs up. A negative patient reaction may be observed by sudden chest movements or jerks, by displaying a negative facial reaction, or by displaying a thumbs down. The operator may adjust the inputted phase shift based on these observed patient reactions. In another embodiment, the operator may select or input a phase shift based on the flow waveform of patient 150 for a non-pressure supported breath.

In some embodiments, the phase shift module 118 applies a phase shift based on generated sensor output. In some embodiments, the phase shift is preprogrammed into the ventilator 100. In some embodiments, as discussed above, the phase shift is preprogrammed into the ventilator 100 based on patient disease state, such as COPD, asthmatic, and/or congestive heart failure. In some embodiments, the ventilator 100 may adjust the phase shift based on generated sensor output such as patient drive, compliance, resistance, or a flow waveform of patient for a non-pressure supported breath.

In an embodiment, where the ventilator 100 adjusts the phase shift based on patient drive an indication of a patient's respiratory drive is made by measuring the pressure generated in the first 100 milliseconds (ms) of inspiration (P₁₀₀)). As discussed above, this calculation is derived from generated sensor output. The higher the P₁₀₀, the greater the respiratory drive. Typically, a high respiratory drive (e.g., P₁₀₀>6 cmH₂O) implies a higher inspiratory flow at an earlier portion of the inspiratory phase. Therefore, in this embodiment, patients exhibiting a high P₁₀₀ are treated with shorter phase shift. In this embodiment, patients with a lower P₁₀₀ (e.g., 2.0 cmH₂O or less) are treated with a longer phase shift.

As discussed above, in patients with a low compliance and/or a high resistance, too fast of a rise time can cause the inspiratory phase to end early. This is the result of a pressure overshoot that causes the ventilator 100 to prematurely detect the end of inspiration. Accordingly, in embodiments where the rise time is adjusted based on compliance or resistance, patient 150 with low compliance or high resistance (e.g., ARDS) are treated with a longer phase shift.

In some embodiments, the ventilator 100 estimates the optimal phase shift from a flow waveform of a non-pressure support breath delivered to the patient 150. By observing the flow waveform for a breath without pressure support, the time between inspiratory trigger and the point at which the peak inspiratory flow is achieved can be measured. This time frame (time between inspiratory trigger and the point at which the peak inspiratory flow is achieved) is utilized by the ventilator 100 to titrate the phase shift in order to align the peak flow associated with shifted pressure profile with that from the non-supported breath.

In other embodiments, the phase shift module 118 applies a phase shift based on operator input and generated sensor output. For example, the ventilator 100 may determine a phase shift or an adjustment of a current phase shift based on generated sensor output and may then recommend this phase shift or adjustment to the operator. However, the ventilator 100 may not apply the recommended phase shift until the recommended phase shift or phase shift adjustment is accepted by the operator based on operator input.

The phase shift module 118 sends the shifted pressure profile to any suitable component of the ventilator 100 for delivery of the shifted pressure profile to the patient 150. In some embodiments, phase shift module 118 sends the shifted pressure profile to the pressure generating system 102, the inspiratory module 104, controller 110, support module 117, and/or the processor 116.

Accordingly, the shifted pressure profile includes phase shift and a pressure profile with a rise time each of which may be adjusted or modified based on operator input and/or generated sensor output. The shifted pressure profile reduces gas leak around the patient interface 180 and patient discomfort (such as gagging) when compared to pressure profiles that do not utilize a phase shift.

In some embodiments, the phase shift module 118 is not activated. In these embodiments, the pressure profile determined by the support module 117 is sent to any suitable component of the ventilator 100 for delivery of the pressure profile to the patient 150. In some embodiments, support module 117 sends the pressure profile to the pressure generating system 102, the inspiratory module 104, controller 110, and/or the processor 116. As illustrated in FIG. 2, the support module 117 and the phase shift module 118 both receive information from the operator input, send information freely between each other, and send information to the pneumatic system 102 in order to deliver the proper and/or desired amount of pressure to the patient 150.

FIG. 3 illustrates an embodiment of a method 300 for ventilating a patient with a ventilator. Method 300 delivers pressure ventilation to a patient on medical ventilator, such as PS or PC ventilation. Method 300 is a method for phase shifting the pressure profile of the pressure based ventilation to provide patient comfort and minimize leaks around the patient interface.

As illustrated, method 300 includes a receiving operation 302. During the receiving operation 302, the ventilator receives a set pressure. The ventilator may receive other parameters, such as a set inspiratory time, respiration rate, ideal body weight, gender, height, weight, age, and/or disease state. These parameters including the set pressure are input by the operator or derived from operator input.

Next, method 300 includes a determining operation 304. The ventilator during determining operation 304 determines a pressure profile based at least on the received set pressure. In some embodiments, ventilator during determining operation 304 determines the pressure profile based additionally on a received respiration rate, ideal body weight, gender, height, weight, age, and/or disease state.

In some embodiments, the ventilator during determining operation 304 adjusts the rise time of the pressure profile based on generated sensor output and/or operator input. In some embodiments, the ventilator during determining operation 304 adjusts the rise time of pressure profile based on operator input. In some embodiments, the ventilator during determining operation 304 determines the inspiratory demand of the patient and adjusts the rise time based on patient demand. Accordingly, in this embodiment, the ventilator during determining operation 304 determines a pressure profile based at least on the received set pressure and the adjusted rise time. The determination of patient inspiratory demand and the adjustment of the rise time based on the determined inspiratory demand are discussed in detail above.

In other embodiments, the ventilator during determining operation 304 determines the resistance and/or compliance of the patient and adjusts the rise time based on the determined resistance or compliance. Accordingly, in this embodiment, the ventilator during determining operation 304 determines a pressure profile based at least on the received set pressure and the adjusted rise time. The determination of resistance and/or compliance and the adjustment of the rise time based on the determined resistance and/or compliance are discussed in detail above.

Further, method 300 includes a shifting operation 306. During the shifting operation 306, the ventilator phase shifts the pressure profile to form a shifted pressure profile. The ventilator during the shifting operation 306 delays the delivery of the pressure profile for a predetermined amount to time to form the shifted pressure profile. In some embodiments, the ventilator during the shifting operation 306 does not increase the pressure delivered to the patient during the predetermined amount of time. In other words, the ventilator during the shifting operation 306 delivers the set PEEP or no pressure to the patient during the predetermined amount of time. In other embodiments, the ventilator during the shifting operation 306 delivers a diminished rise time during the predetermined amount of time. In further embodiments, the ventilator during the shifting operation 306 during a first portion of a predetermined amount of time does not increase the amount of pressure delivered and in second consecutive portion of the predetermined amount of time delivers a diminished rise time to the patient. The first portion and the second portion combine to fill the entire predetermined amount of time.

The ventilator during the shifting operation 306 determines a phase shift based on operator input and/or generated sensor output. In some embodiments, the ventilator during the shifting operation 306 applies a phase shift based on generated sensor output. In some embodiments, the phase shift is preprogrammed into the ventilator. In some embodiments, as discussed above, ventilator during the shifting operation 306 shifts the pressure profile based on a patient disease state, such as COPD, asthmatic, and/or congestive heart failure. In some embodiments, ventilator during the shifting operation 306 phase shifts the pressure profile based on generated sensor output, such as patient drive, compliance, resistance, or a flow waveform of a non-pressure supported breath, which are discussed in more detail above.

In other embodiments, ventilator during the shifting operation 306 determines a phase shift based on operator input and generated sensor output. For example, ventilator during the shifting operation 306 determines a phase shift or an adjustment of a current phase shift based on generated sensor output and may then recommend this phase shift or adjustment to the operator. However, the ventilator during the shifting operation 306 may not apply the recommended phase shift until the recommended phase shift or phase shift adjustment is accepted by the operator based on operator input.

FIGS. 5-7 illustrate example embodiments of shifted pressure profiles that may be determined by the ventilator during the shifting operation 306. Each of FIGS. 5-7 illustrate the implementation of a specific phase shift to three different rise times (Rise Time A, Rise Time B, and Rise Time C). The specific rise time and phase shift utilized by the ventilator during the shifting operation 306 will change based on operator input and/or generated sensor output. In some embodiments, the ventilator and/or the operator will create or choose a shifted pressure profile based on the current patient being ventilated in an attempt to match the current patient's respiratory effort as much as possible with the selected or created shifted pressure profile. In some embodiments, the ventilator during the shifting operation 306 adjust the phase shift applied to the pressure profile between breaths or after a predetermined number of breaths based on operator input and/or generated sensor output. In other embodiments, the ventilator during the shifting operation 306 adjusts the phase shift applied to the pressure profile while being delivered to patient or during the inspiratory phase.

Method 300 also includes a delivering operation 308. During the delivering operation 308, the ventilator delivers the shifted pressure profile to the patient.

In some embodiments, method 300 also includes a phase shift receiving operation 301. The ventilator during the phase shift receiving operation 301 receives operator input to phase shift the pressure profile. In this embodiment, the shifting operation 306 is only performed after ventilator receives operator input to phase shift the pressure profile. In some embodiments, the operator input is the selection of a phase shift button. In other embodiments, the operator input is a specific patient parameter, such as disease state. In some embodiments, the operator input is a disease state of COPD, asthmatic, or congestive heart failure.

In some embodiments, a microprocessor-based ventilator that accesses a computer-readable medium having computer-executable instructions for performing the method of ventilating a patient with a medical ventilator is disclosed. This method includes repeatedly performing the steps disclosed in method 300 above and as illustrated in FIG. 3. In some embodiments, the ventilator system includes means for performing the steps disclosed in method 300 above and as illustrated in FIG. 3.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter.

Numerous other changes may be made which will readily suggest themselves to those skilled in the art and which are encompassed in the spirit of the disclosure and as defined in the claims. While various embodiments have been described for purposes of this disclosure, various changes and modifications may be made which are well within the scope of the present disclosure. Numerous other changes may be made which will readily suggest themselves to those skilled in the art and which are encompassed in the spirit of the disclosure and as defined in the claims.

What is claimed is:

1. A method for ventilating a patient with a ventilator comprising:
receiving a set pressure;
determining a pressure profile based at least on the received set pressure;
phase shifting the pressure profile to form a shifted pressure profile; and
delivering the shifted pressure profile to a patient.

2. The method of claim 1, further comprising:
determining an inspiratory demand of the patient;
adjusting a rise time of the pressure profile based on the inspiratory demand of the patient to form an adjusted rise time,
wherein the step of determining the pressure profile is based at least on the received set pressure and the adjusted rise time.

3. The method of claim 1, wherein the step of phase shifting comprises:
delaying delivery of the pressure profile for a predetermined amount of time after the detection of an inspiration.

4. The method of claim 3, wherein during the predetermined amount of time an amount of pressure delivered to the patient is not increased.

5. The method of claim 3, wherein during the predetermined amount of time an amount of pressure delivered to the patient is increased utilizing a diminished rise time.

6. The method of claim 3, wherein during a first portion of the predetermined amount of time an amount of pressure delivered to the patient is not increased and during a second consecutive portion of the predetermined amount of time the amount of pressure delivered to the patient is increased utilizing a diminished rise time.

7. The method of claim 3, wherein the predetermined amount of time is about 200 ms.

8. The method of claim 1, wherein the step of phase shifting the pressure profile to form the shifted pressure profile is based on a disease state of the patient.

9. The method of claim 1, wherein the step of phase shifting the pressure profile to form the shifted pressure profile is based on at least one of patient drive during P₁₀₀, compliance, and a flow waveform of a non-pressure supported breath.

10. The method of claim 1, wherein the phase shifting is determined based on operator input.

11. The method of claim 1, further comprising:
receiving operator input to phase shift the pressure profile, wherein the step of phase shifting the pressure profile to form the shifted pressure profile is performed in response to the received operator input.

12. A ventilator system comprising:
a pressure generating system adapted to generate a flow of breathing gas;
a ventilation tubing system including a patient interface for connecting the pressure generating system to a patient;
a support module, the support module determines a pressure profile based at least on the received set pressure;
a phase shift module, the phase shift module phase shifts the pressure profile determined by the support module to form a shifted pressure profile,
wherein the pressure generating system delivers the shifted pressure profile to the patient.

13. The ventilator system of claim 12, further comprising:
one or more sensors operatively coupled to at least one of the pressure generating system, the patient, and the ventilation tubing system, wherein the one or more sensors generate output indicative of at least one of an inspiration flow and an inspiration pressure;
wherein the support module determines an inspiratory demand of the patient based on at least one of the inspiration flow and the inspiration pressure;
wherein the support module adjusts a rise time of the pressure profile based on the inspiratory demand of the patient to form an adjusted rise time,
wherein the support module determines the pressure profile based at least on the received set pressure and the adjusted rise time.

14. The ventilator system of claim 12, wherein the phase shift is a delay of delivery of the pressure profile for a predetermined amount of time based on a function of delivered volume, an inspiration time, a percentage of inspiration time, a percentage of delivered volume, and/or a pressure reached.

15. The ventilator system of claim 14, wherein during the predetermined amount of time an amount of pressure delivered to the patient is not increased.

16. The ventilator system of claim 14, wherein during the predetermined amount of time an amount of pressure delivered to the patient is increased utilizing a diminished rise time.

17. The ventilator system of claim 12, wherein during a first portion of a predetermined amount of time an amount of pressure delivered to the patient is not increased and during a second consecutive portion of the predetermined amount of time, the amount of pressure delivered to the patient is increased utilizing a diminished rise time.

18. The ventilator system of claim 12, wherein the phase shift module determines the shifted pressure profile based on at least one of a received disease state of the patient, a P₁₀₀, a compliance, and a determined flow waveform for the patient of a non-pressure supported breath.

19. The ventilator system of claim 12, further comprising a display that displays at least one of the a graph of the phase shift, the pressure profile, a rise time, a diminished rise time, disease state, a P₁₀₀, a compliance, a predetermined amount of time, a flow waveform for the patient of a non-pressure supported breath, and the shifted pressure profile.

20. A computer-readable medium having computer-executable instructions for performing a method of ventilating a patient with a ventilator, the method comprising:
receiving a set pressure;
repeatedly determining a pressure profile based at least on the received set pressure;
repeatedly phase shifting the pressure profile to form a shifted pressure profile; and
repeatedly delivering the shifted pressure profile to a patient.
